(19) United States
(12) Patent Application Publication
(10) Pub. No.: US 2011/0250154 A1
(43) Pub. Date: Oct. 13, 2011

(54) COSMETIC COMPOSITIONS COMPRISING POLYHYDROXYLTATE FATTY ALCOHOLS AND DERIVATIVES AND USES THEREOF

(76) Inventors: Shai Meretzki, Haifa (IL); Gennady Rosenblat, Haifa (IL); Joseph Segal, Haifa (IL)

(21) Appl. No.: 13/062,748

(22) PCT Filed: Sep. 8, 2009
(86) PCT No.: PCT/IL2009/000874

§ 371 Date: Jun. 27, 2011

Related U.S. Application Data
(60) Provisional application No. 61/136,469, filed on Sep. 8, 2008.

(51) Int. Cl. A61K 8/34; A61K 8/67; A61Q 17/04; A61Q 19/08; A61K 8/37; A61Q 19/02

(52) U.S. Cl. 424/59; 514/546; 514/738; 424/62

(57) ABSTRACT

Cosmetic methods and compositions for preventing or reversing damage caused to the skin of a subject by environmental pollution and/or UV radiation, the methods and compositions comprising isolated polyhydroxylated fatty alcohols or derivatives thereof for topical application, in an amount effective to prevent or reverse the harmful effects of environmental pollution and/or UV radiation on the skin e.g. by reducing the damage to the skin cells and/or improving the aesthetic appearance of human skin.

### FIELD OF THE INVENTION

The present invention is generally in the field of cosmetics, and specifically relates to cosmetic compositions and methods for preventing or treating environmental damage and UV induced damage to skin.

### BACKGROUND OF THE INVENTION

The appearance and physical properties of human skin cells, especially keratinocytes, fibroblasts and sebocytes, change with age. Aging occurs as a result of the accumulation of molecular modifications which manifest as macroscopic clinical changes. Factors including ultraviolet radiation (UV), atmospheric pollution, wounds, infections, traumatisms, anoxia, cigarette smoke, and hormonal status have a major role in increasing the rate of accumulation of molecular modifications and thus accelerating the aging process.

The primary intracellular cause of skin aging is oxidation, resulting in generation of free radicals that damage cells and destroy areas of the skin. The skin possesses an elaborate antioxidant defense system to deal with ultraviolet (UV) induced oxidative stress. However, extensive exposure to UV radiation, can overwhelm the cutaneous antioxidant capacity leading to oxidative damage and ultimately to premature skin aging.

Exposure to ultraviolet (UV) irradiation has been found to induce several mediators such as interferon (IFN) gamma and Tumor Necrosis Factor (TNF) alpha, which are believed to be—involved in for both acute skin inflammation and subacute chronic inflammation, the cumulative degenerative effects of which lead to intrinsic and extrinsic aging. Thus, wrinkles, shallowness, and decrease in the mechanical properties and the overall aesthetic appearance of the skin occur over time.

TNF-alpha is considered to be one of the most important tissue factors involved in epidermal damage in response to chronic and acute solar radiation. TNF-alpha is produced by a wide variety of cells, including macrophages, natural killer cells (NK), T lymphocytes, and keratinocytes. It was demonstrated that different stimuli induce TNF-alpha production via different cells and regulatory mechanisms. When the skin is irradiated with UV radiation, TNF-alpha is produced by residual epidermal T lymphocytes (Hassan-Zahraee, M, Wu, J, Gordon, J: “Rapid synthesis of IFN-gamma by T cells in skin may play a pivotal role in the human skin immune system”, Int. Immunol. 2002, 10:1599-1612), and keratinocytes.

At the mRNA level, UV radiation has been shown to up-regulate the level of tumor necrosis factor alpha mRNA in cultured keratinocytes by 20-40 fold within 4 hours.

Recent studies have also indicated that TNF-alpha has significant impact on cell kinetic and DNA repair after exposure to UVB irradiation. UV can directly cause DNA damage, such as formation of Cyclobutane Pyrimidine Dimmers (CPD) and 6-4-photoproducts, both of which can be repaired by the Nucleotide Excision Repair (NER) system (Moriwaki S, Takahashi Y: “Photoaging and DNA repair”, J Dermatol Sci. 2008 June; 50(3):169-76; Bykov V J, Sheehan J M, Hemminki K, Young A R.: “In situ repair of cyclobutane pyrimidine dimers and 6-4 photoproducts in human skin exposed to solar simulating radiation”, J Invest Dermatol. 1999 March; 112(3):326-31). It was demonstrated that TNF-alpha increased the proportion of cycling cells and enhanced the rate of apoptosis, allowing for cells containing un-repaired cyclobutane pyrimidine dimmers (CPD) to enter the cell cycle. In this way, TNF-alpha plays a major role in the promotion of skin photoaging.

Interferon gamma (IFN-γ) is another key cell factor which has been implicated in the development of UV-associated skin remodeling. IFN-γ is secreted by T lymphocytes, dendritic cells and Natural Killer cells in response to various stimuli. A small but significant and sustained production of IFN-γ in epidermal skin exposed to UV radiation was previously reported by Shen et al. (Shen J, Bao S, Reeve V E: “Modulation of IL-10, IL-12, and IFN-gamma in the epidermis of hairless mice by UVA (320-400 nm) and UVB (280-320 nm) radiation.” J Invest Dermatol 113: 1059-1064, 1999). The development of pigmented spots is one of the most representative symptoms of skin photoaging. Among many kinds of pigmented spots formed in the human skin, freckles, melasma, and solar lentigines are most commonly observed. The pivotal role of IFN-γ in the formation of pigmented spots was recently demonstrated by Aoki H and Moro O. “Up-regulation of the IFN-gamma-stimulated genes in the development of delayed pigmented spots on the dorsal skin of F1 mice of HR-1×HR/De” J Invest Dermatol. 2005 May; 124(5):1053-61. Using models of mice dorsal skin, Aoki H and Moro O, have demonstrated a very significant increase of over 4-fold higher concentration of secreted IFN-γ in the UV exposed pigmented lesions compared with the non-irradiated controls.

It has been hypothesized that the initial source of IFN-γ may be residual epidermal T-lymphocytes activated by the UV exposure. The secreted IFN-γ stimulates keratinocytes and up regulates the transcription of Monocytes Chemo-attractant Protein (MCP)-2, interferon-inducible protein (IP)-10, and monokine induced by interferon-γ (MIG), and recruits more T lymphocytes to the damage site. The infiltrated T-lymphocytes produce further chemokines and cytokines, including additional IFN-γ. In addition to the T lymphocytes activity, the inflammatory chemokines and cytokines secreted by those cells and by the activated keratinocytes, also cause infiltration and activation of mast cells, monocytes and macrophages. All these cells are involved in the formation of an interactive network, and provide a suitable local environment for melanocyte activation. Furthermore, IFN-γ synergistically potentiates TNF-alpha-induced activity of Nuclear Factor (NF)-kappa B causing skin aging.

Due to the fact that UV radiation causes T cell proliferation and infiltration of inflammatory induced cells into the damaged skin that is an important step in the inflammatory process, T cells inhibitors were developed to delay this process and prevent skin injury. Currently, a number of T lymphocyte inhibitors, and TNF-alpha, and IFN-γ expression inhibitors, are known and protein-based TNF alpha inhibitors have demonstrated efficacy and have been approved for clinical use in various inflammatory diseases. However, all these compounds are potent drugs and cause systemic potentially serious adverse effects and cannot be used in cosmetic formulations (Palladino M A, Bahjat F R, Theodorakis E A, Moldawer L L: “Anti-TNF-alpha therapies: the next generation”, Nat Rev Drug Discov. 2003 September; 2(9):736-46). An example of the use of interferon antagonists for the treatment of interferon related inflammation disease is described in U.S. Pat. No. 7,285,526. An example of the use of interferon antagonists for the treatment of specific skin conditions characterized by increased T cell activation, e.g. UV damage is disclosed in U.S. Pat. No. 7,323,171.

A limited number of natural compounds with inhibition effect on T cells proliferation and TNF alpha and IFN expression have been described (Drug Discov Today. 2006 August; 11(15-16):725-32).

The avocado fruit is widely consumed as food through the world, and is also used for various medicinal and cosmetic purposes. The health benefits of avocado may be due to the fact that it contains over 20 essential nutrients and various potentially biologically active compounds.

Acetyl derivatives of Polyhydroxylated Fatty Alcohols (PFA) are present both in the pear and in the seeds of avocado fruit. Non-acetylated polyhydroxylated fatty alcohol was also detected in a minor quantity in the avocado fruit. Acetyl derivatives of polyhydroxylated fatty alcohols are a group of lipids having relatively similar structures. These substances have been previously found to be active against cancer cell lines (Oberlies N H, Rogers L L, Martin J M, and McLaughlin J L: “Cytotoxic and insecticidal constituents of the unripe fruit of Persea Americana.” J Nat Prod 1998; 61:781-5), and have demonstrated liver protective and anti toxic activity (Kawagishi H, Fukumoto Y, Hatakeyama M, He P, Arimoto H, Matsuzawa T, et al. “Liver injury suppressing compounds isolated from avocado.” J Agric Food Chem 2001; 49:2215-21) and moderate activity against epimastigotes and trypomastigotes (Abe F, Nagafuji S, Okawa M, Kinjo J, Akahane H, Ogura T, et al.: “Trypanocidal constituents in plants 5. Evaluation of some Mexican plants for their trypanocidal activity and active constituents in the seeds of Persea Americana”. Biol Pharm Bull 2005; 28:1314-7). In addition, some of these compounds have also demonstrated antifungal (Dominguez F, Helms G L, Prusky D, Browse J.: “Antifungal compounds from idioblast cells isolated from avocado fruits”. Phytochemistry 2000; 54:183-9) and antibacterial properties (Neeman I., Lishitz, A. and Kashman, Y. 1970.: “New antibacterial agent isolated from avocado pear.” Applied Microbiology, 19: 470-473), and significant ability to inhibit acety CoA carboxylase activity (Hashimura H, Ueda C, Kawabata J, Kasai T.: “Acetyl-CoA carboxylase inhibitors from avocado (Persea americana Mill.) fruits”. Biosci Biotechnol Biochem 2001; 65:1656-8).

The unsaponifiable fraction of avocado oil is the fraction of fatty substances, which remain insoluble in water after prolonged hydrolysis in alkaline solution, and could be extracted using organic solvents. The unsaponifiable fraction of avocado seed oil is useful in several cosmetic and therapeutic applications. For example, PCT Application WO 99/43298 describes the use of a dermatological formulation containing unsaponifiable lipid extract from avocado seed for ameliorating stretch marks and keratoses. During the process of triglyceride isolation by saponification of avocado oil in mineral alkaline solution, acetyl derivatives of polyhydroxylated fatty alcohols were found to be hydrolyzed to non-acetylated polyhydroxylated fatty alcohol and to remain in that state in the unsaponifiable fraction.

Non-acetylated polyhydroxylated fatty alcohols may also be detected in saponified (hydrolyzed) extract of avocado seed where their concentration may comprise up to 10% of the unsaponifiable fraction. Another main group of compounds present in avocado seed-unsaponifiables are furans (FIG. 4), which may be present at a concentration of up to 30% of the unsaponifiable fraction. These furan compounds have previously demonstrated biological active properties.

U.S. Pat. No. 6,582,688 describes a method for isolation of avocado fractionation of unsaponifiable substances which allows the separation of the fraction consisting of furan lipids in a mixture with non-acetylated polyhydroxylated fatty alcohols (up to 25%) and the use of those furan based compounds, in cosmetic treatment of the skin and for treating inflammatory disorders. The presence in these cosmetic applications of both polyhydroxylated fatty alcohols—in deacetylated form, and the furan lipids are the major disadvantage of this formulations.

Furan lipids are known potent inhibitors of lysyl oxidase (M. J. Werman, S. Mokady, and I. Neeman: “Partial Isolation and Characterization of a New Natural Inhibitor of Lysyl Oxidase from Avocado Seed Oil”, J, Agric. Food Chem. 1990, 38, 2164-2168; Rosenblat G, Kagan H, Shah M, Spiteller G, Neeman I: “Chemical Characterization of Lysyl Oxidase Inhibitor from Avocado Seed Oil”, JAOCS 72, 225-229 (1995)), an enzyme which is important for normal skin tone and elasticity.

Due to the inhibitory effect of furans on lysyl oxidase activity, furan-containing lipids might serve as an anti-fibrotic drugs in the treatment of diseases involving excess collagen and elastin deposition, in scleroderma-related conditions for the inhibition of intra and intermolecular cross-linking, and possibly from enhancing the cleavage of newly-formed cross-links. In contrast, in other conditions reduced lysyl oxidase activity is associated with increased risk of skin laxity and joint hyper-extensibility (Song Y L, Ford J W, Gordon D: “Shanley C J Regulation of lysyl oxidase by interferon-gamma in rat aortic smooth muscle cells.” Arterioscler Thromb Vasc Biol. 20 (2000), 982-988). Using experimental animal models, it was demonstrated that in vivo reduced lysyl oxidase activity is associated with the risk of development of aortic aneurysms (Maki J M, Rasanen J, Tikkanen H, Sormunen R, Makikallio K, Kivirikko K I, Soininen R.: “Inactivation of the lysyl oxidase gene Lox leads to aortic aneurysms, cardiovascular dysfunction, and prenatal death in mice.” Circulation. 2002 Nov. 5; 106(19):2503-9).

### SUMMARY OF THE INVENTION

There is thus a need for, and it would be useful to have, a method and cosmetic compositions for preventing and/or reversing damage caused to the skin of a subject as a result of exposure to environmental pollution and to UV radiation.

The present invention thus provides cosmetic methods and compositions for preventing or reversing damage caused to the skin of a subject by environmental pollution and/or UV radiation, the methods and compositions comprising isolated polyhydroxylated fatty alcohols or derivatives thereof for topical application, in an amount effective to prevent or reverse the harmful effects of environmental pollution and/or UV radiation on the skin e.g. by reducing the damage to the skin cells and/or improving the aesthetic appearance of human skin.

By “cosmetic” it is meant any composition or method which improves the visual appearance of the skin, including but not limited to the appearance of enhanced pliability, softness and elasticity; wrinkle reduction; and/or reduced evidence of the aging process; or a combination thereof. The term “the aesthetic appearance of the skin” refers to the visual appearance of the skin.

By any of ameliorating, preventing, treating or reversing skin aging, it is meant any of ameliorating, preventing, treating or reversing one or more visual effects of aging on the skin, including but not limited to cuticle thickening, rough texture, wrinkles, flaccidness, loss of elasticity, pigmented spots (including but not limited to freckles, melasma, and solar lentigines), fragile skin, sagging skin, fine lines, thinning skin, lack-luster skin, fatigued skin, and dry skin or mottled pigmentation, or a combination thereof.

In case of UV induced damage, such damage relates to any visual effect of UV radiation on the skin, including but not limited to cuticle thickening, rough texture, wrinkles, flaccidness, loss of elasticity, or mottled pigmentation, or a combination thereof. Therefore, ameliorating, preventing, treating or reversing UV induced damage relates to ameliorating, preventing, treating or reversing any of the above effects of UV induced damage, or a combination thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, “about” means plus or minus approximately ten percent of the indicated value.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A and 1B are gas chromatography elution profiles of the natural derivative of polyhydroxylated fatty alcohols from avocado seed (A) and pear (B);

FIG. 2 shows the structures of major natural derivatives of polyhydroxylated fatty alcohols;

FIG. 3 illustrates the chemical structure of the main de-acetylated derivatives of natural polyhydroxylated fatty alcohols from avocado;

FIG. 4 illustrates the chemical structure of representative furan lipids from avocado;

FIG. 5 is a bar chart demonstrating the inhibitory effect of natural polyhydroxylated fatty alcohols on the proliferation of primary human T cells and Jurkat cells;

FIG. 6 is a bar chart demonstrating the effect of natural derivative of polyhydroxylated fatty alcohols and de-acetylated polyhydroxylated fatty alcohols on the viability of primary human T-cells;

FIG. 7 is a bar chart demonstrating the inhibitory effect of natural derivatives of polyhydroxylated fatty alcohols from avocado on TNF-α and IFN-γ secretion by human activated CD3+ T lymphocytes;

FIGS. 8A and 8B are bar charts demonstrating the protective effect of polyhydroxylated fatty alcohols on HaCaT cells (A) and primary human keratinocytes (B) viability after exposure to UVB irradiation;

FIG. 9 is a bar chart demonstrating the effect of polyhydroxylated fatty alcohols on 12-O-Tetradecanoylphorbol-13-acetate (TPA) (also known as phorbol 12-myristate 13-acetate, PMA)-induced loss of mitochondrial potential;

FIG. 10 is a bar chart demonstrating the effect of polyhydroxylated fatty alcohols on TPA-induced IL-6 secretion by primary human keratinocytes;

FIG. 11 is a bar chart showing the effect of polyhydroxylated fatty alcohols on UV-induced IL-6 secretion by primary human keratinocytes;

FIG. 12 is a bar graph showing the effect of polyhydroxylated fatty alcohol on UVB-induced PGE₂ secretion by primary human keratinocytes;

FIG. 13 is a table demonstrating the inhibitory effect of polyhydroxylated fatty alcohols and its mixture with ursolic acid and acetyl salicylic acid on prostaglandins (PGE₂) secretion by UV-irradiated primary human keratinocytes;

FIG. 14A features photographs of sunburn cells in UVB-irradiated skin explants with or without PFAs (shown as “polyol”);

FIG. 14B is a bar chart showing protective effect of polyhydroxylated fatty alcohols against the formation of sunburn cells in UVB-irradiated skin explants;

FIG. 15 is a bar chart showing the effect of PFA to promote removal of UVB-induced cyclopyrimidine dimmers (CPD) in primary human keratinocytes; and

FIG. 16 is a bar chart showing the effect of polyhydroxylated fatty alcohols on viability of keratinocytes (HaCaT cells) (A) and fibroblasts (B).

### DESCRIPTION OF THE INVENTION

Polyhydroxylated fatty alcohols (PFA) have significant biological effects on human skin cells and can influence the properties of skin, confirming their usefulness in improving the aesthetic appearance of the skin and for prevention or amelioration of the effects of skin aging. Various embodiments of the present invention comprise or use these compounds. By “polyhydroxylated fatty alcohols” it is meant any polyhydroxylated fatty alcohol which may be found in or derived from substances found in any type of fruit or vegetable, preferably avocado seed or the flesh of the avocado fruit. By “derived from”, it is meant any type of derivation of any polyhydroxylated fatty alcohol which may be found in any type of fruit or vegetable, preferably avocado seed or the flesh of the avocado fruit, as described herein.

Furthermore, these polyhydroxylated fatty alcohols are preferably also able to reduce or prevent skin cell damage caused by environmental causes; for example they can reduce and repair skin damage caused by UV radiation exposure and may be used for a variety of cosmetic applications.

In addition, in at least some embodiments, these polyhydroxylated fatty alcohols are able to prevent or reduce the harmful effects to the skin caused by direct damage to the skin cells (keratinocytes) and by activation of inflammatory cells and production of pro-inflammatory mediators, following skin damage by UV or other environmental pollutions, for various cosmetic applications in order to improve the visual appearance of the skin. The above description in the Background, regarding the effects of UV damage and aging on the deep tissue of the skin is also related to the biological effects in the stratum corneum, including elasticity, barrier properties and permeability, and the like.

The present inventors have surprisingly discovered that polyhydroxylated fatty alcohols, for example those isolated from avocado and avocado seed, have significant cosmetic effects on human skin and biological effects on the skin cells, thereby confirming their usefulness for amelioration of the effects of skin aging, and improving the aesthetic appearance thereof. Furthermore, these polyhydroxylated fatty alcohols are preferably also able to reduce skin cell damage, for example caused by toxic compounds or by UV radiation exposure.

In addition, polyhydroxylated fatty alcohols are able to affect proliferation and activity of inflammatory cells following UV radiation exposure and/or environmental damage, and reduce the production of pro-inflammatory mediators, and can be used to reduce skin local inflammatory reaction for cosmetic applications.

In addition, in at least some embodiments, these polyhydroxylated fatty alcohols showed no negative skin responses in clinical study making it possible to use for a variety of cosmetic applications.

Without wishing to be limited by a single hypothesis, such an improvement in the visual appearance of skin may optionally occur through the biological effects of PFAs on the skin cells, more preferably including both the stratum corneum and the deeper tissues of the skin and/or through the functions of the polyhydroxylated fatty alcohols as sphingolipid-like compounds, hence providing a more naturally compatible solution.

The present inventors have isolated and quantified the active ingredients present in avocado seed and avocado fruit, for example using gas-chromatographic and HPLC methods.

According to some embodiments of the present invention, there is provided a topical cosmetic composition comprising polyhydroxylated fatty alcohols or derivatives thereof in an amount effective to improve the aesthetic appearance of human skin. According to further features in preferred embodiments of the present invention, the derivative of polyhydroxylated fatty alcohols substantially comprises an acetylated derivative.

According to some embodiments, the polyhydroxylated fatty alcohols or derivatives thereof are isolated from natural sources.

Optionally and preferably, the isolated natural polyhydroxylated fatty alcohols are isolated from a fruit or vegetable source, such as, for example, avocado fruit or avocado seed.

According to still further features in the described preferred embodiments, the natural polyhydroxylated fatty alcohols are isolated in substantially pure form.

As used herein, “natural” includes all materials from the extract, including acetylated forms, with a minor component of non-acetylated. After hydrolysis the fatty alcohols become de-acetylated and are not referred to as natural herein.

According to some embodiments, the polyhydroxylated fatty alcohols or derivatives thereof are synthetic products.

According to some embodiments, the polyhydroxylated fatty alcohols or derivatives thereof are synthetic products.

According to still further features in the described preferred embodiments, the polyhydroxylated fatty alcohols are present in a concentration of from about 80% to about 95% w/w of the isolated material.

According to still further features in the described preferred embodiments, the polyhydroxylated fatty alcohols are present in a concentration of from about 0.001% to about 20% w/w of the total composition.

According to still further features in the described preferred embodiments, the composition is free or completely free of furan lipids. By “completely free of furan lipids” it is meant that up to about 5% of furan lipids may be present in the composition. By “free of furan lipids” it is meant that up to about 20%, preferably up to about 15%, more preferably up to about 10% and most preferably up to about 7.5% of furan lipids may be present in the composition.

According to still further features in the described preferred embodiments, the polyhydroxylated fatty alcohols or derivatives thereof are substantially selected from the group consisting of, 1-Acetoxy-2,4-dihydroxy-16-heptadecene, 1-Acetoxy-2,4-dihydroxy-16-heptadecyne.

The present invention further provides the use of any of the compositions described hereinabove for use in reducing external signs of UV-induced aging in human skin and skin cells.

The present invention further provides the use of any of the compositions described hereinabove for prophylactic treatment of UV damage to human skin and skin cells for improving the visual appearance of skin.

Hence, in at least some embodiments, the polyhydroxylated fatty alcohols mixture of the present invention can be administered as preventive compounds before and/or after the exposure to UV radiation.

The present invention further provides the use of any of the compositions described hereinabove for use in preventing external signs of aging, including but not limited to UV-induced aging in human skin, such as, for example, the appearance of pigmented spots (including but not limited to freckles, melasma, and solar lentigines), fragile skin, sagging skin, fine lines, wrinkles, thinning skin, lack-luster skin, fatigued skin, and dry skin.

According to some embodiments, the external signs of aging are due to UV-induced premature aging. The present invention further provides the use of any of the compositions described hereinabove for use in reducing redness or irritation of the skin.

According to some embodiments, the composition of the present invention is used for reducing redness or irritation on the skin, such as that caused, for example, by rosacea or telangiectasia associated with rosacea, and/or due to UV induced and/or environmental damage. An example of environmental damage is diaper rash, such that the composition is optionally used for prevention and/or relief of diaper rash.

According to some embodiments of the present invention, there is provided a method for the isolation of natural polyhydroxylated fatty alcohols and derivatives thereof from a fruit or vegetable source, the process comprising crushing fruit or vegetable source to obtain a lyophilized powder; extracting the lyophilized powder with a non-polar organic solvent or polar solvent to obtain a crude lipid extract; concentrating the crude lipid extract using a non-polar or polar solvent to obtain a concentrated crude lipid extract; separating the polyhydroxylated fatty alcohols and derivatives thereof from the concentrated crude lipid extract by crystallization at a temperature lower than room temperature to obtain crystals; filtering the crystallized polyhydroxylated fatty alcohols and derivatives thereof; adding ethanol to the filtrate; filtration of insoluble material remaining in the ethanol; evaporation; and re-crystallization with a non-polar solvent.

Ultraviolet radiation is harmful to a wide range of biological systems. The extent of damage depends upon the level and duration of exposure as well as the susceptibility and resistance of the exposed organism. The key human health effects from exposure to UV radiation include skin cancer, cataracts, and immunosuppression. In addition, other dermatological effects include severe photo-allergies and accelerated aging of the skin. Damage to the skin by UV radiation reduces its immunological defenses, impeding resistance to infectious diseases as well as to skin tumors, and diminishing the effectiveness of vaccines. The compositions of the present invention are preferably able to prevent and/or ameliorate the negative effects of ultraviolet radiation on the skin.

The natural polyhydroxylated fatty alcohol mixture of the present invention demonstrated significant inhibition of the inflammatory reaction characterized by expression of inflammatory mediators and by activated T cells proliferation. Testing the active compound activity in vitro on cells damaged by UV radiation showed that the compounds can inhibit UV induced IL-6 expression and PGE2 expression.

In addition, in vitro biological studies of the polyhydroxylated fatty alcohols of the present invention have demonstrated that the active compound could significantly protect viability of irradiated skin cells, inhibit UV induced keratinocytes death and inhibit sunburn cell formation in skin explants, and remarkably promote the removal of UVB-induced cyclopyrimidine dimmers (CPD) in human keratinocytes. The protective mechanisms of the polyhydroxylated fatty alcohols are still under examination.

Furthermore, it has surprisingly been demonstrated that acetylated derivatives of polyhydroxylated fatty alcohols from avocado seed have significantly less toxicity compared to non-acetylated polyhydroxylated fatty alcohols, which are isolated from the same source and formed in the process of saponification.

In vitro toxicological studies have shown that polyhydroxylated fatty alcohols at concentrations in which they demonstrated high biological activity (up to 1 μg/ml) are not toxic to keratinocytes, fibroblasts, or T cells. These results, as well as additional findings demonstrated that polyhydroxylated fatty alcohols are found not only in the seed but also in the eatable parts of the avocado fruit, enabled human safety analysis of the active compound.

Safety testing of the polyhydroxylated fatty alcohols was carried out to identify whether the compounds of the present invention cause any undesirable reaction when coming in contact with the skin. The Repeat Insult Patch Test is considered the most reliable study for predicting the likelihood of an ingredient or a product of producing contact hypersensitivity in human subjects. The protocol used ten continuous patches on 50 volunteers, none of which exhibited adverse skin reactions.

Thus, it would clearly be highly beneficial to provide the active ingredients from avocado seeds for cosmetic applications which are free or substantially free of such furan compounds to avoid the possible negative effect of the compounds. Similarly, using the natural polyhydroxylated fatty alcohol is much more preferable instead of their deacetylated derivatives to avoid said effect, which is associated with increased cytotoxicity of the compounds.

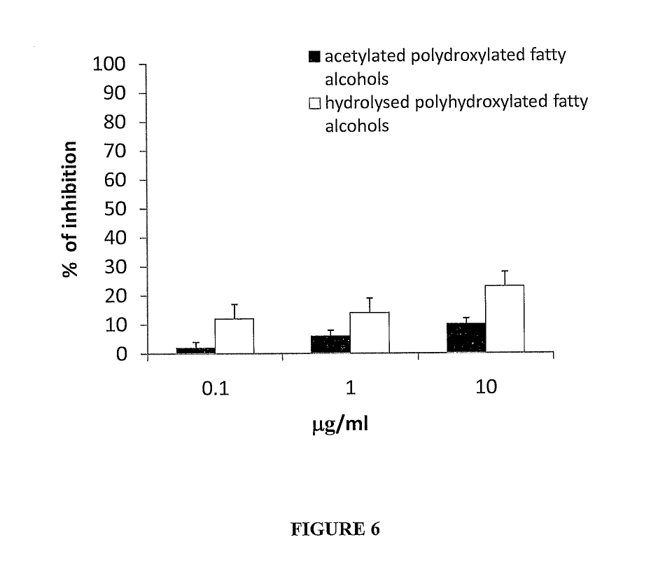

Alkaline hydrolysis of polyhydroxylated fatty alcohols leads to formation of mainly 1,2,4-trihydroxy heptadec-16-en and 1,2,4-trihydroxy heptadec-16-yn. Hydrolyzed polyhydroxylated fatty alcohol significantly increase the toxicity of the compounds (FIG. 6). Hence, there is a significant advantage to using naturally occurring acetylated polyhydroxylated fatty alcohols instead of de-acetylated compounds separated from unsaponifiables, or in combination with other avocado unsaponifiables as described in PCT Application WO 99/43298 and U.S. Pat. No. 6,582,688. Moreover, the presence at least one acetyl group in the structure of derivatives of polyhydroxylated fatty alcohols increases the stability of the product and protects the active molecule against oxidation.

Cosmetic Formulations

According to at least some embodiments of the present invention there is provided cosmetic formulations for topical administration.

According to some embodiments, the composition is in any form for conventional skin external preparations, including but not limited to facial cosmetics such as lotion, milky lotion, cream, and packs; makeup cosmetics such as pre-makeup, foundation, cheek color, lipstick, lip cream, eye shadow, eye liner, mascara, and sunscreen; body cosmetics; aromatic cosmetics; skin cleansers such as makeup remover, facial cleanser, and body shampoo; an aerosol, cake, cream, ointment, emulsion, essence, foam, gel, lotion, mousse, paste, patch, pencil, serum, solution, towelette, mask, body wrap, spray, or stick.

The cosmetic composition may comprise, for example, an oil/water emulsion, such as one based on ammonium acryloyl-dimethyltaurate/VP-copolymer; a gel formulation, such as one based on glyceryl polymethacrylate/propylene glycol; or a non-ionic emulsion, such as one based on ethoxylated fatty alcohols and sorbitol fatty acid esters.

The preferred composition for topical administration is a non-ionic oil-in-water emulsion. Emulsion composition preferably comprises from about 30% to about 80% (w/w) oil phase, most preferably from about 35% to about 40% (w/w). The water phase is preferably in the range of from about 19% to about 70% (w/w) and most preferably from about 59% to about 65% (w/w). The concentration of the polyhydroxylated fatty alcohol is preferably from about 0.01% to about 5% (w/w) and most preferably from about 0.1% to about 1%.

The oil phase is preferably comprised of at least one of natural vegetable oils, polyunsaturated fatty acids, vitamin A, E, and F, ascorbyl palmitate, anti-oxidants and other suitable components, or mixtures thereof. The natural vegetable oils are preferably limited to jojoba oil, wheat germ oil, avocado oil, soybean oil, sesame oil, rice oil, or any other suitable natural vegetable oil. The water phase of the cream composition is preferably comprised of a mixture of water, natural plant extracts, humectants, non-ionic emulsifiers, and other suitable components.

The cream formulation can be optionally administrated by topically applying onto the facial skin, neck, scalp, around the eyes and to the body skin, preferably before exposure of the face and body skin to a hostile environment of toxins, pathogens, LTV radiation, negative environmental factors and pollutants.

The lipophilic natural oil composition preferably comprises at least one of a polyhydroxylated fatty alcohol, natural vegetable oils, polyhydroxylated fatty acids, vitamin A, E, and F, antioxidants, penetration enhancers and other suitable components. Polyhydroxylated fatty alcohols are preferably present in a concentration in the range of from about 0.01% to about 5% (w/w) and more preferably from about 0.1% to about 1% (w/w). The concentration of natural vegetable oil is preferably from about 20% to about 80% (w/w) and most preferably from about 65% to 75% (w/w).

The natural vegetable oils are preferable comprised of one or more of wheat germ oil, sesame oil, soybean oil, avocado oil, olive oil and rice oil. The fatty acids are preferably one or more of linoleic acid, linolenic acid, omega 3, gamma linolenic and arachidonic acid—omega 6 or mixture thereof. The penetration enhancers are preferably present in a concentration of from about 0.5% to about 2.0% (w/w) and most preferably in a concentration of 2% (w/w).

Preferred penetration enhancers include, but are not limited to, propylene glycol dipelargonate and ethoxydiaglycol. The propylene glycol dipelargonate is preferably present in a concentration of about 1.60% (w/w) and ethoxydiaglycol is preferably present in a concentration of about 0.4% (w/w). Additional suitable components are optionally present in a range of from about 19% to about 80% (w/w).

The lipophilic natural oil formulation can be optionally administered by topically applying onto the facial skin, neck, lips, scalp, around the eyes and on the body skin, preferably twice daily. Application is especially preferable before exposure of the face and body skin to a hostile environment of toxins, pathogens, UV radiation, negative environmental factors and pollutants.

Optionally, the composition further comprises a cosmetically acceptable carrier. Examples of suitable carriers include water; plant oils, such as jojoba oil, avocado oil or corn oil; mineral oils; esters such as octyl palmitate, so isopropyl myristate and isopropyl palmitate; ethers such as dicapryl ether, diethylene glycol monoethyl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, dimethicone, dimethicone cross-polymer, polysiloxanes and their derivatives, preferably organomodified derivatives; hydrocarbon oils such as mineral oil, petrolatum, isoeicosane and polyisobutene; polyols such as propylene glycol, glycerin, butylene glycol, pentylene glycol, 1,3-butylene glycol, and hexylene glycol; waxes such as beeswax and botanical waxes; or any combination or mixtures thereof.

The carrier may optionally further comprise an anti-oxidant, such as, for example, green tea extract, ginger extract and grape seed extract, or mixtures thereof.

The composition may further optionally comprise one or more cosmetically acceptable excipients, including but not limited to water soluble colorants (such as FD&C Blue #1); oil soluble colorants (such as D&C Green #6); chelating agents (such as Disodium EDTA); emulsion stabilizers (such as carbomer); preservatives (such as Methyl Paraben); fragrances (such as pinene); flavoring agents (such as sorbitol); humectants (such as polyethylene glycol, propylene glycol, glycerin, 1,3-butylene glycol, hexylene glycol, xylitol, sorbitol, maltitol, chondroitin sulfuric acid, hyaluronic acid, mucoitin sulfuric acid, caronic acid, Atelocollagen, cholesteryl 12-hydroxystearate, sodium lactate, bile salts, dl-pyrrolidonecarboxylic acid salts, short chain soluble collagen, diglycerol (ethylene oxide) propylene oxide adduct, extract of chestnut rose, extract of malifoils (Achillea millefolium), and extract of melilots); whitening agents (such as placental extract, glutathione, extract of creeping saxifrage (Saxifrage stolonifera), waterproofing agents (such as PVP/Eicosene Copolymer); water soluble film-formers (such as Hydroxypropyl methylcellulose); oil-soluble film formers (such as Hydrogenated C-9 Resin); cationic polymers (such as Polyquatemium 10); anionic polymers (such as xanthan gum); emollients, such as dimethicone, polysilicones and cyclomethicone; lubricants; moisturizers; skin penetration enhancers; surfactants; thickeners; viscosity modifiers; and the like.

The effective amount of the isolated natural polyhydroxylated fatty alcohols or derivatives thereof and the duration of application of the composition will vary with the particular sign of aging being reduced or prevented, the age and physical condition of the person, the extent of the sign of aging, the particular carrier utilized, and like factors in the knowledge and expertise of those skilled in the art. The duration of application may be, for example, once or twice a day for a period of at least one week, two weeks or more.

According to still further features in the described preferred embodiments, the composition of the present invention further comprises an additional compound selected from the group consisting of oil soluble vitamins, antioxidants and antioxidant extracts of polyphenols, natural plant extracts and natural seed oil, such as, for example, one or more of green tea based polyphenols, epigallocatechin (EGC), epicathechin 3-gallate (ECG), epicatechin gallate (EG), silymarines, grape extract, resveratrol Ginkgo biloba extract, polyphenols, rosmarinic acid, ursolic acid, Coenzyme Q10 (coQ10), glutathione, vitamin C, Vitamin A, Lycopene, Carotenoids, Flavonoids/polyphenols vitamin E, Alovera extract, and pomegranate seed oil.

The additional compound may comprise, for example, one or more of anesthetics; anti-allergenic; antimicrobials; antifungals; natural or synthetic COX (cyclo-oxygenase) inhibitors such as acetyl salicylic acid or ursolic acid; antiseptics; chelating agents; colorants; depigmenting agents; emollients, such as dimethicone, polysilicones and cyclomethicone; exfollients, such as retinal, retinal and retinoic acid; fragrances; emulsifiers; humectants; insect repellents; lubricants; moisturizers; preservatives; skin penetration enhancers; stabilizers; sunscreens; surfactants; thickeners; viscosity modifiers; antiseptics; antioxidants and vitamins. However, it should be noted that any such one or more additional compound is included for its cosmetic effect(s) on the visual appearance of the skin.

According to some embodiments, the composition of the present invention optionally further comprises synthetic and natural compounds selected from the group including but not limited to the oil soluble vitamins group, antioxidants or antioxidant extracts containing of polyphenols, such as green tea based polyphenols, epigallocatechin (EGC), epicathechin 3-gallate (ECG) and epicatechin gallate (EG), silymarines, grape extract, resveratrol, natural plant extracts like Ginkgo biloba extract, aloe vera extract and natural see oil like pomegranate seed oil.

Examples of suitable vitamins include vitamin A, vitamins of the B group, vitamin C, vitamin E, and derivatives thereof. Useful derivatives include retinal, retinal, retinoic acid, and other related compounds having retinoid or retinoid-like activity, magnesium ascorbyl phosphate, sodium ascorbyl phosphate, tocopheryl ascorbyl phosphate, and ascorbyl tocopheryl maleate.

Examples of antioxidants capable of slowing or preventing the oxidation process include compounds such as green tea based polyphenols, Coenzyme Q10 (CoQ10), glutathione, vitamin C, Vitamin A, Lycopene, Carotenoids, Flavonoids/polyphenols and vitamin E as well as enzymes such as catalase, and peroxidase.

Other particularly useful additional ingredients are sunscreens. Preferred sunscreens are those with a broad range of UVB and UVA protection, such as octocrylene, avobenzone (Parsol 1 78 9), octyl methoxycinnamate, homosylate, benzophenone, camphor derivatives, zinc oxide, and titanium dioxide. Other particularly useful additional ingredients are exfoliating agents, such as alphahydroxyacids, betahydroxyacids, oxa acids, oxa diacids, and their derivatives such as esters, anhydrides and salts thereof.

### Polyhydroxylated Fatty Alcohols and Derivatives

The present invention therefore provides topical cosmetic compositions comprising natural isolated polyhydroxylated fatty alcohols or derivatives thereof, or synthetic versions thereof, in an amount effective to improve the aesthetic appearance of human skin.

According to preferred embodiments, polyhydroxylated fatty alcohols or derivatives thereof preferably comprise a backbone of from C13 to C25 carbons, optionally with at least one unsaturated carbon bond. Preferably, if at least one unsaturated carbon bond is present, it is present between the last two carbons of the backbone, whether as a double bond or triple bond. Optionally and more preferably, the hydroxyl groups are present at C1, C2 or C4.

According to other preferred embodiments, derivatives of polyhydroxylated fatty alcohols preferably comprise polyhydroxylated fatty alcohols that have been acylated (esterified) or oxidized or have undergone reaction of the unsaturated carbon bonds with one or more other molecules, for example for hydrogenation of the unsaturated carbon bonds.

The term “natural derivatives of polyhydroxylated fatty alcohols” as used herein refers to all types of derivatives of polyhydroxylated fatty alcohols which are present in fruit or vegetable extracts and which have not undergone hydrolysis. As noted above, the major component is acetylated while a minor component is deacetylated.

The term “acetylated polyhydroxylated fatty alcohols” as used herein refers to all types of derivatives of polyhydroxylated fatty alcohols containing at least one acetyl group instead of a hydrogen atom in a hydroxyl group. The acetyl group may optionally be at C1, C2 or C4, but is preferably at C1 or C4.

According to some embodiments, the polyhydroxylated fatty alcohols are natural polyhydroxylated alcohols, isolated in substantially pure form, such as, for example, 95% pure, 90% pure, 85% pure or 80% pure.

According to some embodiments of the present invention, the isolated natural polyhydroxylated fatty alcohols or derivatives, or synthetic equivalents thereof include but are not limited to 1-Acetoxy-2,4-dihydroxy-16-heptadecene or 1,2-dihydroxy-4-acetoxy-16-heptadecene, 1-Acetoxy-2,4-dihydroxy-16-heptadecyne or 1,2-dihydroxy-4-acetoxy-16-heptadecyne or combinations thereof.

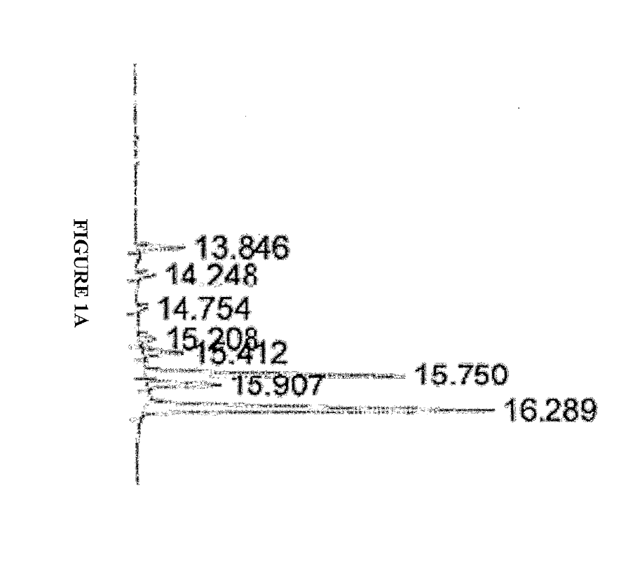

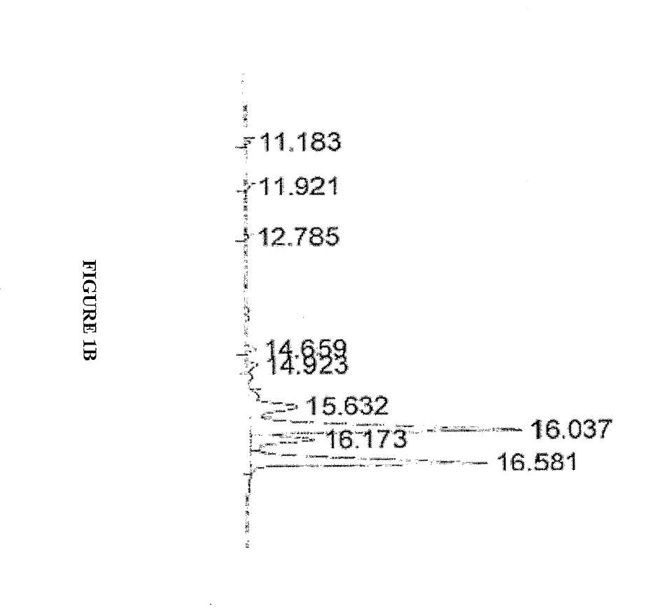

The elution profile of natural acetylated polyhydroxylated fatty alcohols that were separated from avocado seed and fruit are shown in FIGS. 1A and 1B. The structure of main derivatives of natural acetylated fatty acid that were separated from avocado seed and pear are shown in FIG. 2.

Identification of the compounds was performed by using GC/MS and LC/MS analysis. Elucidation of the chemical structure of the compounds was based on the similarity of characteristic peaks (m/e) in Mass spectra and molecular peak of sodium adduct [M⁺Na]⁺ of the compounds with that which were described earlier for corresponding polyhydroxylated fatty polyhydroxylated fatty alcohols.

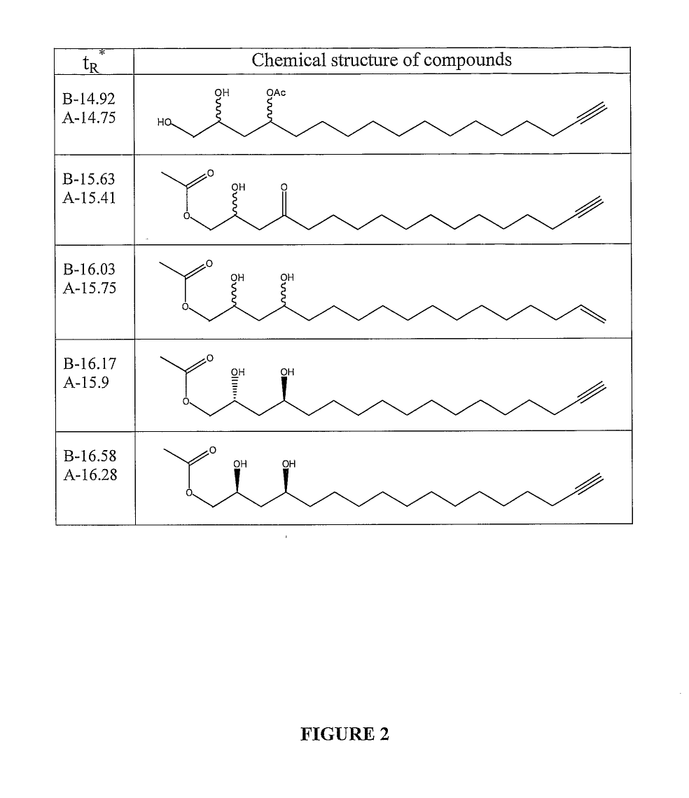

FIG. 3 shows representative structures of de-acetylated polyhydroxylated fatty alcohols obtained by saponification of acetylated polyhydroxylated fatty alcohols in alkaline solution.

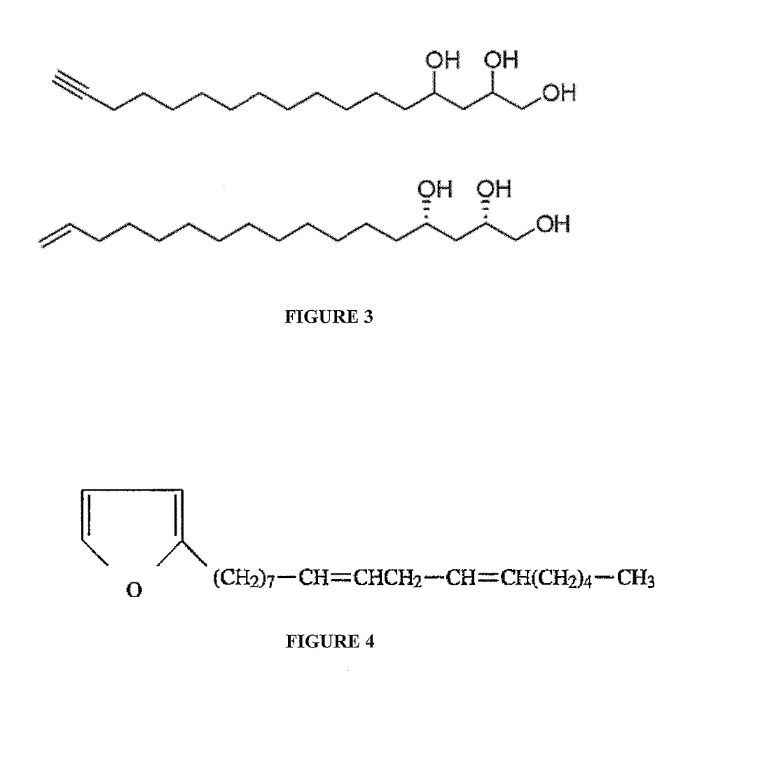

FIG. 4 demonstrated the structure of representative furan lipids from avocado seed, thereby showing the differences in structure from the preferred embodiments of polyhydroxylated fatty alcohols of the present invention.

According to some embodiments, there is provided a method for specific isolation of the fraction of natural derivatives of polyhydroxylated fatty alcohols from a crude extract of avocado seed. The process optionally and preferably includes the stages of isolating the avocado seeds from the avocado fruit, crushing, and lyophilizing the avocado seeds.

The lyophilized powder is extracted using a non-polar (organic) solvent (e.g. hexane, petroleum ether) or polar solvent (ethanol, methanol), to obtain the crude lipid extract.

The crude lipid extract is concentrated by using a non-polar solvent (hexane, petroleum ether) or polar solvent (ethanol, methanol).

The desired components are preferably separated from the concentrated the crude extract by methods of cool crystallization, i.e. crystallization at a temperature, which is lower than room temperature, followed by filtration.

Filtered compounds are dissolved in ethanol, and the insoluble, highly non-polar compounds are separated by filtration.

The ethanol is evaporated and the compounds obtained are re-crystallized with a non-polar solvent, such as, for example, hexane—or petroleum ether

Surprisingly, it was found that the compounds that are re-crystallized from cooled avocado extract in a non-polar solvent are enriched with acetylated polyhydroxylated fatty alcohols and do not include furan containing lipids, or contain those compounds only a minor trace amount.

This method of isolation of natural derivatives of polyhydroxylated fatty alcohols significantly increases the concentration of these active compounds, by at least about four times, compared to background art methods using molecular distillation, such as described in U.S. Pat. No. 6,582,688, which results in a concentration of up to 25% polyhydroxylated fatty alcohols in a mixture with furan containing lipids.

The natural derivatives of polyhydroxylated fatty alcohols separated by this method may comprise up to 95% by weight dry powder.

The composition of the present invention may optionally comprise from about 0.01% to about 90% by weight of natural polyhydroxylated fatty alcohols, or synthetic equivalents thereof.

The present invention further provides a method for the isolation of natural polyhydroxylated fatty alcohols and derivatives thereof which protect against environmental damage, UVB photo damage, and TPA-induced loss of mitochondrial potential, and are inhibitors of T lymphocyte proliferation, TNF alpha and IFN-γ expression, from a fruit or vegetable source which comprises avocado fruit and/or avocado seeds.

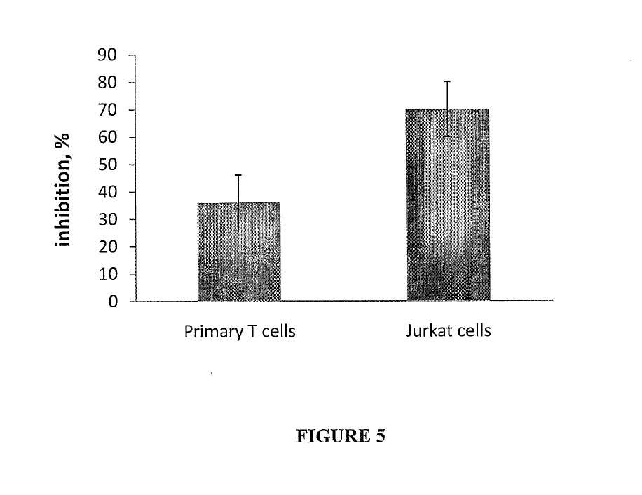

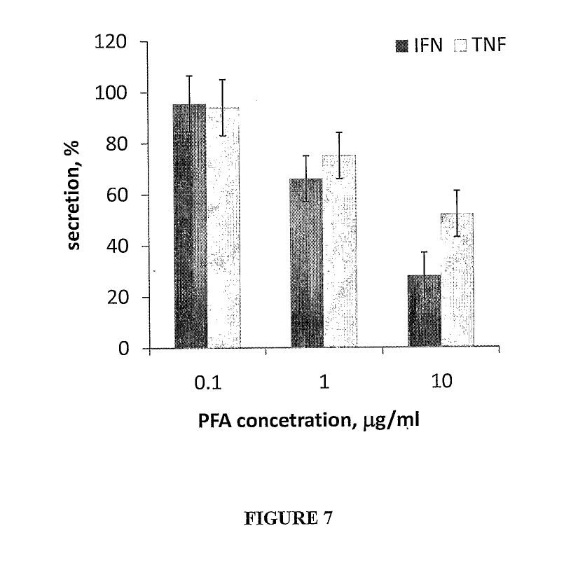

The ability of polyhydroxylated fatty alcohols to inhibit T lymphocyte proliferation, TNF alpha, and IFN gamma expression has been demonstrated in vitro as demonstrated in FIGS. 5 and 7.

During the process of triglyceride isolation by saponification of avocado oil in mineral alkaline solution, acetyl derivatives of polyhydroxylated fatty alcohols were found to be hydrolyzed to non-acetylated polyhydroxylated fatty alcohol and to remain in that state in the unsaponifiable fraction. By checking T cell viability in the presence of deacetylated fatty alcohols, the present inventors have demonstrated that deacetylation of polyhydroxylated fatty alcohols significantly increase the toxicity of the isolated compounds (FIG. 6).

In some embodiments, the present invention also optionally comprises de-acetylated fatty alcohols, for example prepared with hydrolysis, such that the uses described herein for natural polyhydroxylated fatty alcohols may also be ascribed to such de-acetylated alcohols.

The composition of polyhydroxylated fatty alcohols of the present invitation can be used for protection of the skin and skin cells against damage caused by exposure to pollutants and to prevent skin aging. Examples of pollutants include, but not limited to atmospheric factors, chemical pollutants, and biological pollutants. Examples of atmospheric factors that affect the skin include but not limited to radiation such as UV radiation from the sun, ozone, acid rain, and extreme temperatures. Chemicals and biological pollutants include pollutants from cars, industry, free radicals, cleaning materials, cosmetics.

Exposure of the subject to pollutants can result in development of abnormal skin conditions, such as skin aging, skin irritation, and low skin humidity. The polyhydroxylated fatty alcohols mixture of the present invention can be administered as preventive compounds before and after the exposure to any of the aforementioned pollutants.

Dosing is dependent on the responsiveness of the subject to the polyhydroxylated fatty alcohol. Polyhydroxylated fatty alcohol composition is effective in an amount of from about 0.01 μg/ml to about 10 μg/ml. The formulation containing the polyhydroxylated fatty alcohols preferably contains from about 0.01% to about 5% polyhydroxylated fatty alcohols and more preferable from about −0.1% to about −1%. However higher or lower doses are possible. The dose frequency of dosing would be dependent on the responsiveness of the subject. A person of ordinary skill in the art can easily determine optimum dosages, dosing methodologies and repetition rates.

### EXAMPLES

Reference is now made to the following examples, which together with the above description, illustrate the invention in a non limiting fashion.

### I: Isolation of Polyhydroxylated Alcohols

### Example 1

### Isolation of Natural Polyhydroxylated Fatty Alcohols from Avocado Seeds and their Alkaline Hydrolysis

Avocado seeds were separated from the avocado pear followed by freezing and lyophylization. 10 kg of lyophilized and powdered seed was consequently extracted using hexane in Soxhlet apparatus for 14 h.

Organic solvents were evaporated in a rotor evaporator at temperature intervals of 40-60° C., at a pressure of about 30 millibar. Extracted compounds were re-dissolved with two volumes of hexane or petroleum ether (as a non-limiting example of a non-polar solvent) and then were put into a cold room having a temperature in the range of 2-8° C. for about 12 hours for the process of cool crystallization.

Crystallized compounds were separated from the solvent by filtration in Worthman filter paper.

The process yielded 30 g of crystalloid compounds. Elution profile and chemical structure according to GC/MS and HPLC/MS-ECI analysis are presented in FIGS. 1A and 2. No furan lipids were detected.

De-acetylated PFAs were obtained by alkaline hydrolysis of natural PFA in 2% sodium hydroxide in methanol at room temperature for 24 h. At the end of the reaction, the solution was neutralized by 5% hydrochloric acid and de-acetylated polyhydroxylated fatty alcohols were extracted by diethyl ether. The solvent was removed under reduced pressure.

A gas-chromatographic method was developed for the quantification of the needed polyhydroxylated fatty alcohols. The analysis was performed using a Perkin Elmer Gas Chromatograph, equipped with flame ionization detector. A mixture of polyhydroxylated fatty alcohols separated from avocado seed by cool crystallization was used as a standard.

### Example 2

### Isolation of Polyhydroxylated Fatty Alcohols from Avocado Pear

In order to isolate polyhydroxylated fatty alcohols from the edible part of the avocado fruit, 200 g ground avocado pear (Hagalil or Ettinger) were extracted twice in 400 ml heated ethanol at 60° C. for 1 h followed by acetone extraction at 4° C. overnight. All extracts were collected and the solvents were evaporated. Dried extract was re-dissolved in 35 ml hexane. Avocado pear hexane extract was refrigerated (4° C.) overnight and precipitated polyhydroxylated fatty alcohols were separated by filtration and the sample was analyzed by GC column. The process yielded 100-140 mg of crystalloid compounds. Elution profile by GC and chemical structure according to GC/MS analysis are presented in FIG. 1B and FIG. 2.

### Example 3

### Effect of Polyhydroxylated Fatty Alcohols on T-Cells and Jurkat Cells Proliferation and T-Cells Viability

Human T cells were purified from the peripheral blood of healthy human donors. The whole blood was incubated (20 min, 22° C.) with RosetteSep™ human T-cell enrichment cocktail (StemCell Technologies, Vancouver, BC, Canada). The remaining unsedimented cells were then loaded onto Lymphocyte Separation Medium (ICN Biomedicals; Belgium), isolated by density centrifugation, and washed with PBS. The purified cells (>95% CD3⁺ T cells) obtained were cultured in RPMI containing antibiotics and 10% heat-inactivated FCS. Proliferation of T-cells was assessed by the 2,3-bis[2-methoxy-4-nitro-5-sulfophenyl]-2H-tetrazolium-5-carboxanilide (XTT) assay after mitogenic anti-CD3 cells activation in presence PFA. For the study of the effect of polyhydroxylated fatty alcohols on T-cells viability CD3 T cells were incubated with the reagents for 72 hours and then, T-cells viability was determined by XTT assay.

Activation of CD3⁺ T-cells by addition of anti-CD3 stimulates T-cells proliferation. This proliferation, nevertheless, was inhibited by pretreatment of the cells with PFA. About 40% growth inhibition was observed in anti-CD3 activated T-cells that were treated with PFA at concentration of 10 μg/ml. Jurkat cells were more susceptible to PFA, achieving more than 80% inhibition of cell proliferation in similar condition. The results are shown in FIG. 5.

Viability assay revealed slightly decreased number of T-cells—after 72 h administration with PFA, compared with non-treated cells (FIG. 6). At PFA concentration of 10 μg/ml, the number of viable cells was decreased by about 10%-, that did not account the 40% of T-cells inhibition proliferation.

De-acetylated PFA at concentration of 10 μg/ml significantly decrease T-cell proliferation (about 20% inhibitions) (FIG. 6).

### Example 4

### Inhibitory Effect of Natural Derivatives of Polyhydroxylated Fatty Alcohols from Avocado Seeds, on TNF Alpha and INF Gamma Production by T Lymphocytes

The following experiment was performed in order to confirm the inhibitory effect of natural derivatives of polyhydroxylated fatty alcohols from avocado seeds, on TNF alpha and INF gamma production by activated T lymphocytes.

T cells (2×10⁶ cells per ml) were activated (1 hr, 37° C.) with the indicated concentrations of reagents in 24-well plates in media based on RPMI containing 10% heat-inactivated FCS. The cells were washed, and re-plated at the same concentration on anti-CD3 mAb pre-coated 24-well plates (2 μg/ml; non tissue culture grade plates) at 4° C. for 24 hr with and without polyhydroxylated fatty alcohols from avocado seeds. The supernatants were collected, and the cytokines content (TNF-α, IFN-γ) were determined by using ELISA commercial kits (OptiEIA kits; BD Pharmingen), —according to the manufacturer's instructions.

Pre-treatment of the cells with PFA caused significant dose-dependent suppression of TNF alpha and IFN gamma secretion by T-cells. TNF alpha and IFN gamma secretion by T-cell pre-treated with PFA at concentration 1 μg/ml was 25% and 30% below than that in control cells. Suppression of cytokine expression in presence of PFA at concentration of 10 μg/ml was much higher achieving of about 50% and 70% correspondingly. The results are shown in FIG. 7.

### Example 5

### Effect of Polyhydroxylated Fatty Alcohols on Keratinocytes Cell Viability after Exposure to UVB Irradiation

HaCaT, a human immortalized keratinocytes cell line, was grown at 37° C. in 5% CO2 in DMEM (Biological Industries, Beit Haemek, Israel) supplemented with 10% fetal calf serum, L-glutamine 2 mM and antibiotics (100 U penicillin per ml and 100 mg streptomycin per ml). The cell line originated from the laboratory of N. Fusenig, Heidelberg, Germany. Only early passages (<50) were used for the experiments

Primary normal human epidermal keratinocytes were provided by M. Chaouat M. Sc. (Laboratory of Experimental Surgery, Hadassah Hospital, Jerusalem, Israel)

The cells were isolated from the skin removed during cosmetic plastic surgery. Briefly, a thin split thickness skin biopsy is taken asceptically and trypsinized overnight at 4-8° C. The epidermal layer is separated from the dermal layer and the single cells are isolated and cultured in specialized keratinocytes medium. The keratinocytes are then redistributed into the flasks containing lethally irradiated 3T3. The flasks are incubated at 37° C. 8-10% CO₂. Upon reaching subconfluency the cells are redistributed to new flasks without the 3T3 feeder layer.

HaCaT Cell viability was measured by MTT assay. The cells were inoculated in 96 well Microtitre plates at the concentration of 1×10⁵ cells/well. Before UVB irradiation, the cells were pre-treated with PFA for 60 min. After removing the media, the cultures were washed thoroughly with PBS, filled with PBS and irradiated with UVB 30 mJ/cm2. After cell irradiation, PBS saline was changed to growth medium containing corresponding PFA concentration and cells were incubated for more 24 hours.

At the end of incubation time, 100 μl of MTT (5 mg/ml) was added to each well. After 4 h of incubation at 37° C., the plate was centrifuged at 1000 rpm for 50′. The supernatant was removed and precipitate was dissolved in 100 μl of isopropanol. The O.D of the resulting solution was measured spectrophotometrically at 540 nm.

Viability of primary human keratinocytes was measured using Dead/Live kit (Molecular Probes, Eugene, Oreg., U.S.A.) containing fluorescent dye. Keratinocytes were growth on eight-well g-slides (tissue culture treated, IBIDI) up to sub-confluent condition. Before UVB irradiation, the cells were pre-treated with PFA for 60 min. After removing the media, the cultures were washed thoroughly with PBS, filled with PBS, and irradiated with UVB 20 mJ/cm2. After cell irradiation, PBS saline was changed to growth medium containing corresponding PFA concentration and cells were incubated for more 24 hours. The cell number was calculated by using confocal microscopy.

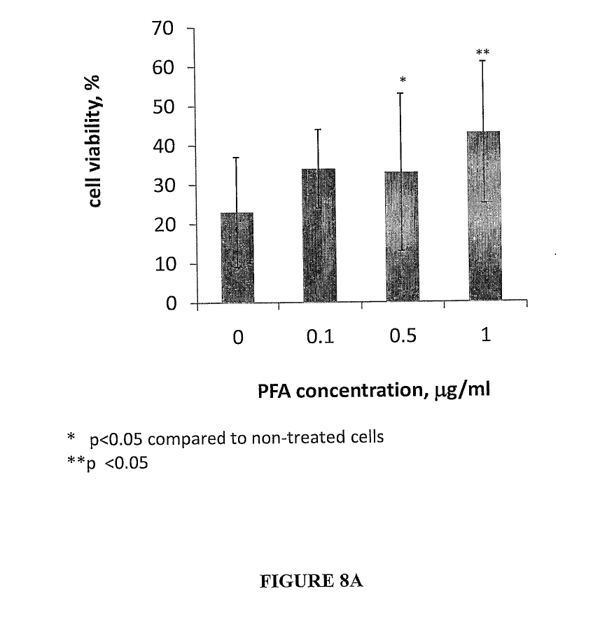

Cell viability is demonstrated in FIG. 8. Cell number is represented as the percentage of the living cells in each sample compared to the initial cells counted. The viability of the control cells in the absence of UV treatment is given as an arbitrary 100%.

As shown in FIG. 8 (A) HaCaT cells exposed to UVB irradiation exhibited 23% cell viability compared to the controls. However, the cell viability was significantly increased when the cell were pretreated with PFA. In those conditions the cell survival increased from 23% to 34% and to 43% after pre-treatment with 0.2 ug/ml and 1 ug/ml PFA correspondingly.

Similarly, to the behavior of the HaCaT cell, pretreatment with PFA significantly increase the viability of UVB exposed primary human keratinocytes, as is demonstrated in FIG. 8(B). In those conditions, the viability of UV-irradiated primary human keratinocytes pre-treated with PFA increased from 50% (for non-treated cells) up to 90%. PFA does not demonstrates any properties of UVB—screeners in wavelengths between 280-320 nm (data hot shown)

### Example 6

### Effect of Polyhydroxylated Fatty Alcohols on TPA-Induced Loss of Mitochondrial Potential (Δψm)

The effect of polyhydroxylated fatty alcohols on UVB- or PMA-induced loss of mitochondrial trans-membrane potential was examined. HaCaT cells were grown for 48 h on μ-slides 8-well (tissue culture treated, IBIDI, Munchen, Germany) and then were pre-treated with polyhydroxylated fatty alcohols for 40 min. PFA treated cells were treated with additional administration of 12-O-Tetradecanoylphorbol-13-acetate (TPA) (also known as phorbol 12-myristate 13-acetate (PMA), at a concentration of 25 ng/ml for 90 min. The cells were washed with PBS and stained with potential-sensitive dye TMRM.

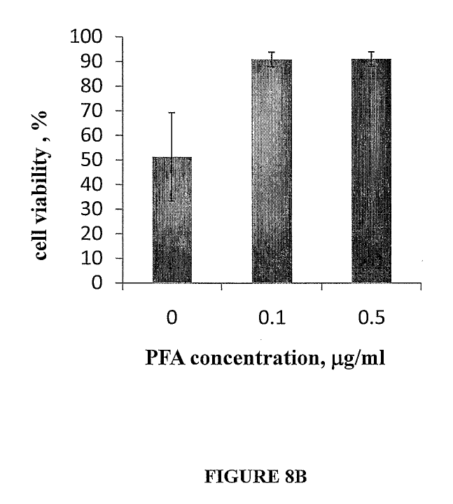

Fluorescence of intra-mitochondrial TMRM was measured by confocal microscopy and trans-membrane potential (Δψm) was expressed in arbitrary units of fluorescence. The effect of polyhydroxylated fatty alcohols on TPA-induced loss of mitochondrial potential is demonstrated in FIG. 9. Pre-treatment of the cells with polyhydroxylated fatty alcohols at concentration ranges of 0.2 μg/ml-1 μg/ml significantly protected HaCaT cells from the loss of mitochondrial potential.

### Example 7

### Effect of Polyhydroxylated Fatty Alcohols from Avocado Seeds on UV- and TPA-Induced IL-6 Secretion in Primary Human Keratinocytes

In order to study the effect on TPA-induced secretion of IL-6, primary keratinocytes at sub-confluent conditions in 24-well plate were treated with polyhydroxylated fatty alcohols, in growth medium for 60 min. At the end of the time, the growth medium was additionally supplemented with 1 ng/ml TPA and the cells were incubated at 37° C., 5% CO₂ for 8 hours. IL-6 was quantified in the growth medium by ELISA method using a commercial kit (Human IL-6 Quantikine HS ELISA Kit, R&D system, MN, U.S.A.), according to the manufacturer's instructions.

For investigation of the effect of UV radiation, primary human keratinocytes at sub-confluent conditions in 24-well plate were treated with polyhydroxylated fatty alcohols in the growth medium for 60 min. After removing the media, the cultures were washed thoroughly with PBS, filled with a 1-cm layer of PBS, and irradiated with UVB (30 mJ/cm²). After the cell irradiation, PBS saline was changed to growth medium containing a corresponding concentration of polyhydroxylated fatty alcohols, and cells were incubated at 37° C., 5% CO₂ for 8 hours. IL-6 was quantified in cell growth medium by ELISA method by using the same kit, according to the manufacturer's instructions.

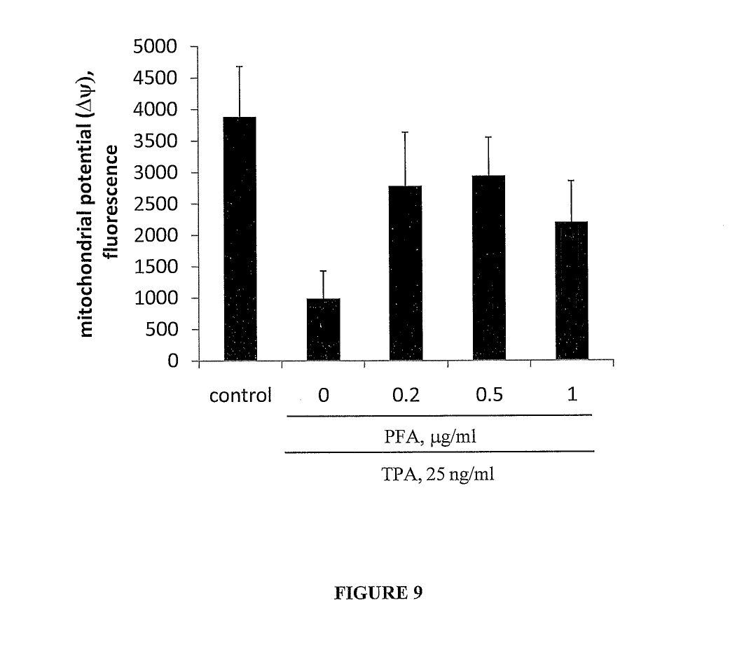

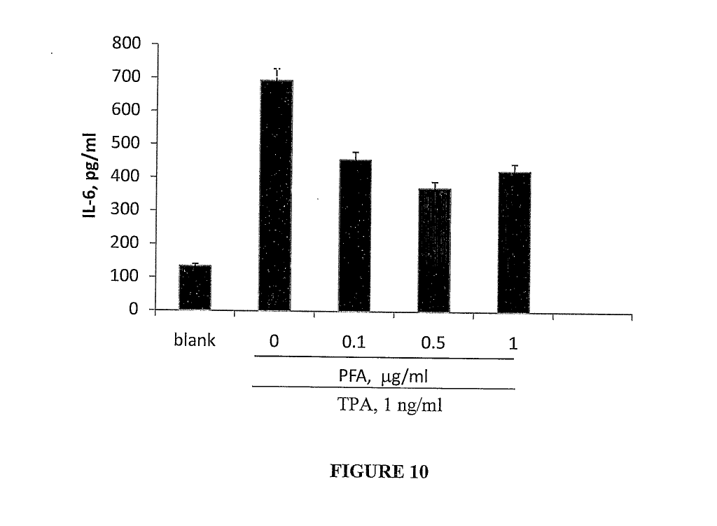

Results are shown in FIGS. 10 and 11. As can be seen, both TPA and UVB radiation induces increased secretion of IL-6 in primary human keratinocytes. Pre-treatment of the cells with PFA significantly inhibited the additional secretion of IL-6 by 40-50%. The difference between PMA-treated and UVB-exposed cells was that the inhibitory effect of PFA on IL-6 was already not apparent at concentration 1 ug/ml and higher in UVB irradiated cells.

### Example 8

### Effect of Polyhydroxylated Fatty Alcohols from Avocado Seeds on UVB-Induced PGE2 Secretion in Primary Human Keratinocytes

Primary human keratinocytes at sub-confluent conditions in 24-well plate were treated with PFA in growth medium for 60 min. After removing the media, the cultures were washed thoroughly with PBS, filled with a 1-cm layer of PBS, and irradiated with UVB (30 mJ/cm²). After irradiation of the cells, PBS saline was changed to growth medium containing corresponding concentrations of polyhydroxylated fatty alcohols, and the cells were incubated at 37° C., 5% CO₂ for 8 hours. PGE2 was quantified in medium by ELISA method by using commercial kit (Prostaglandin E2 Parameter Assay Kit, R&D system, MN, U.S.A.), according to the manufacturer's instructions.

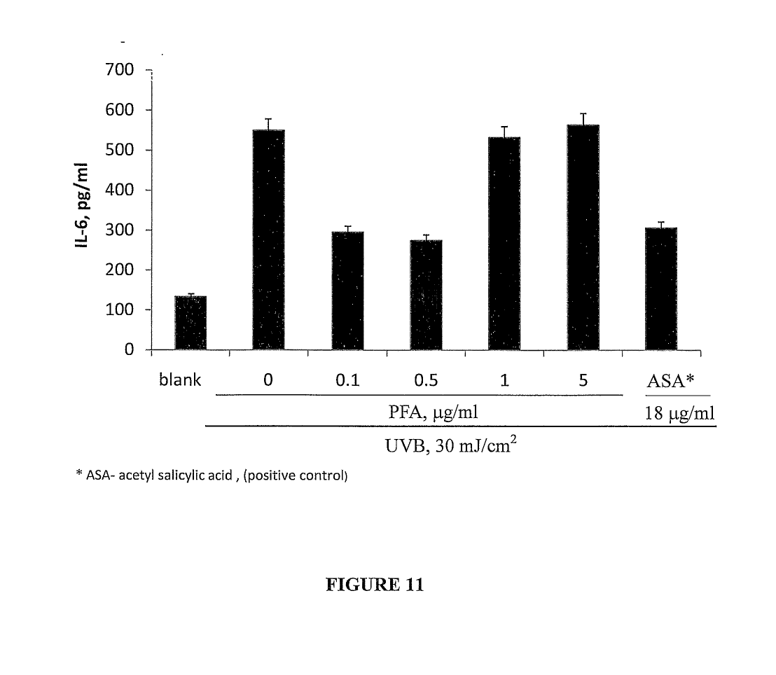

FIG. 12 demonstrates UVB radiation-induced secretion of PGE2 in primary human keratinocytes. Pre-treatment of the cells with polyhydroxylated fatty alcohols inhibits the additional secretion of PGE2. The results presented in FIG. 12 demonstrate the inhibitory effect of polyhydroxylated fatty alcohols on prostaglandin E2 synthesis in UVB exposed keratinocytes.

### Example 9

### Inhibitory Effect of Polyhydroxylated Fatty Alcohol and its Mixture with Ursolic Acid or Acetyl Salicylic Acid on PGE2 Secretion in UV-Irradiated Primary Human Keratinocytes

Primary human keratinocytes at sub-confluent conditions in 24 well plate were treated with ethanol solution of polyhydroxylated fatty alcohol or ursolic acid or acetyl salicylic acid, or with a mixture of polyhydroxylated fatty alcohol and ursolic acid or acetyl salicylic acid in growth medium based on DMEM for 60 min. After removing the media, the cultures were washed thoroughly with PBS, filled with a 1-cm layer of PBS, and irradiated with UVB (30 mJ/cm²). After irradiation of the cells, PBS saline was changed to growth medium containing the test compounds or their mixtures and cells were incubated at 37° C., 5% CO₂ for 8 hours.

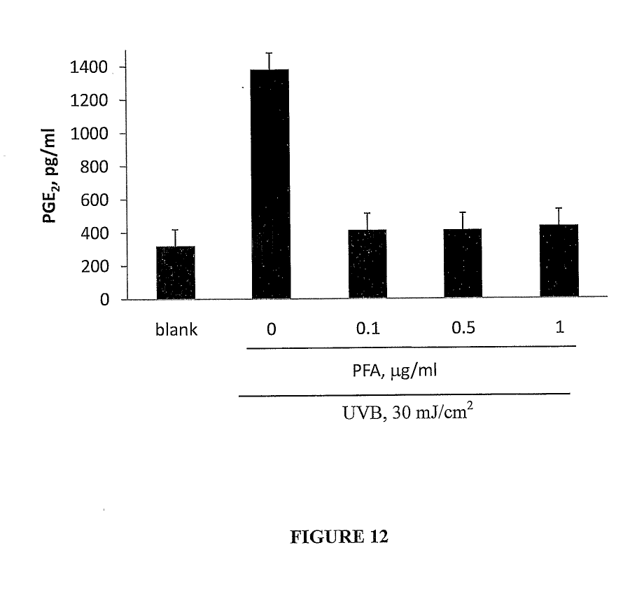

PGE2 was quantified in medium by ELISA method by using the same kit, according to the manufacturer's instructions. The results are shown in FIG. 13

As FIG. 13 demonstrates, there is a synergetic effect between the biological activities of polyhydroxylated fatty alcohols and COX inhibitors such as acetyl salicylic acid or ursolic acid. The mixtures of polyhydroxylated fatty alcohols with COX inhibitors were found to decrease PGE2 secretion by primary human keratinocytes at higher level, comparing to the PGE2 secretion inhibitory ability of any ingredient alone.

### Example 10

### Polyhydroxylated Fatty Alcohol Ability to Protect Skin Tissue from UV Damage in Organ Culture Model

Human skin tissue samples were obtained within 2 hr of surgery (under approval no. 0273-08-HMO of the Institutional Review Board Committee of Hadassah Hospital, Jerusalem, Israel). Organ cultures were prepared essentially as described by Hasson and coworkers (Hasson, E., Slovatizky, Y., Shimoni, Y., Falk, H., Panet, A. & Mitrani, E. “Solid tissues can be manipulated ex vivo and used as vehicles for gene therapy”. J Gene Med. 7: 926-935, 2005). In brief, skin tissue samples were washed in DMEM and cut with a microtome (tissue sectioner, Sorvall model TC-2; Thermo Fisher Scientific, Waltham, Mass.) into thin slices (300 μm) and incubated in DMEM containing 10% FCS, gentamicin (15.2 μg/ml), and Ciproxin (ciprofloxacin, 19 μg/ml) at 37° C., 5% CO₂.

Before UV radiation, each sample was treated for 60 min with polyhydroxylated fatty alcohols. After this time, the media was removed, the cultures were washed thoroughly with PBS, filled with a 1-cm layer of PBS and irradiated with UVB (90 mJ/cm²). After cell irradiation, PBS saline was changed to DMEM—medium containing corresponding concentration of the tested compound. 24 hours later, skin samples were fixed for 30 min in phosphate-buffered saline (PBS) containing 4% paraformaldehyde, rinsed, and embedded in TissueTek (Sakura, Japan). Histology was performed by preparing 8-μm-thick frozen sections using a Young CM-3000 cryostat. Staining was done with hematoxylin/eosin (H&E) according to routine histological methods. Sun burn cells were derived from keratinocytes. Hematoxylin-eosin staining showed a shrunken chromatin, an eosinophilic cytoplasm, and a pyknotic nucleus.

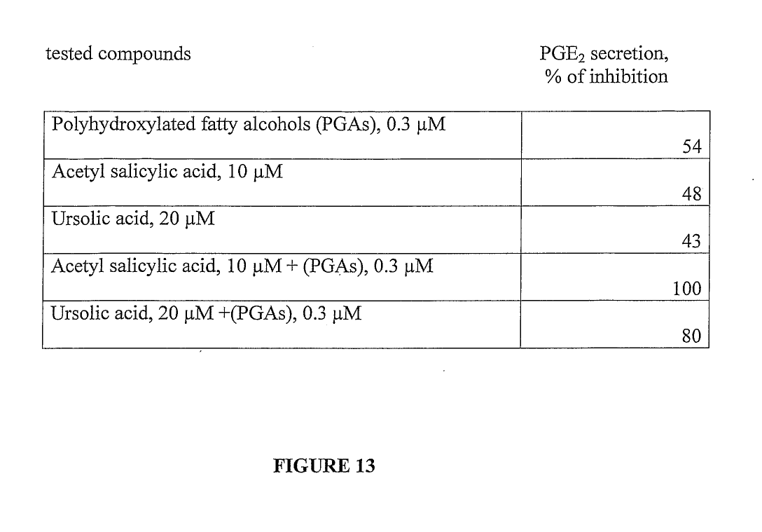

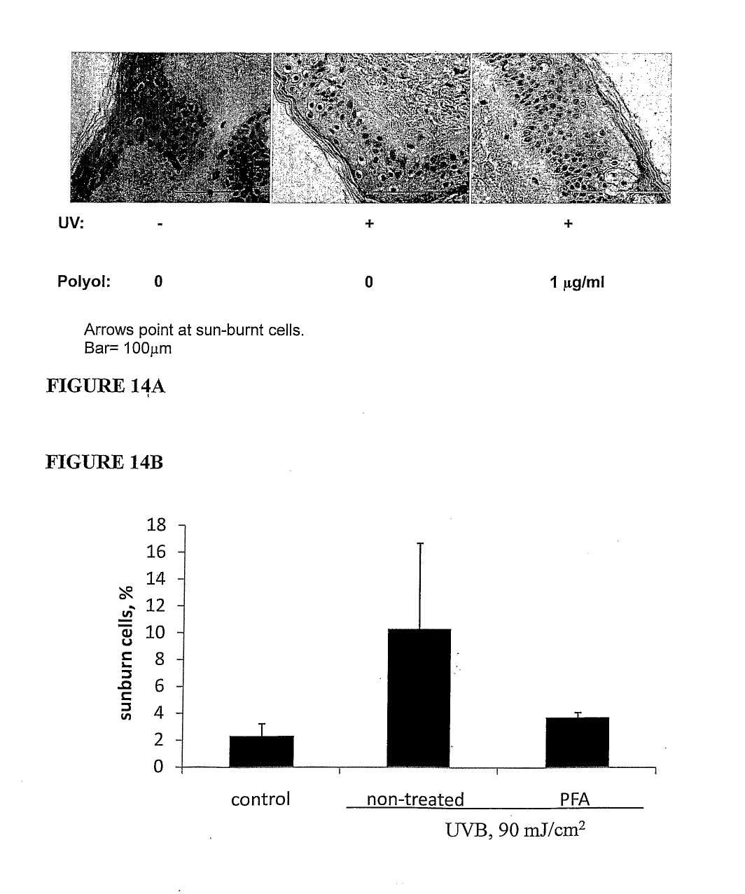

The results are shown in FIGS. 14A and 14B. Pretreatment skin explants with 1 μg/ml polyol dramatically decreased the amount of sunburn and apoptotic cells within the treated and UV-exposed skin culture.

### Example 11

### Effect of PFA to Promote Removal of UVB-Induced CPD in Human Keratinocytes

Primary human keratinocytes at sub-confluent conditions in 9-cm culture dishes were treated with polyhydroxylated fatty alcohols in growth medium for 60 min. After removing the media, the cultures were washed thoroughly with PBS, filled with a 1-cm layer of PBS, and irradiated with UVB (20 mJ/cm²). After the cell irradiation, PBS saline was changed to growth medium based on DMEM, containing corresponding polyhydroxylated fatty alcohols concentration and cells were incubated at 37° C., 5% CO₂ for 24 hours followed by DNA extraction using Wizard Genomic DNA Purification Kit (Promega, Madison, Wis., USA). [12].

In the control samples, the DNA was extracted immediately after irradiation. In brief, 5 μg/well denatured DNA was applied, in triplicate, into poly-L-lysine (Sigma) pre-coated ELISA plates, washed 5 times with PBS and blocked with 2% FCS in PBS. As first antibody, an anti-thymidine dimer H3 clone 4F6 (Affiteck, Oslo, Norway) diluted 1:1,000 in 2% FCS in PBS was used. As second antibody, a biotin-SP-conjugated goat anti-mouse Ig diluted 1:50,000 was used, followed by peroxidase-conjugated strepavidin (Jackson) diluted 1:10,000. The peroxidase reaction was performed using 0.4 mg/ml OPD (o-phenylamine) (Sigma) in the presence of 0.02% H2O2, and color intensity was measured by spectrophotometry at 492 nm.

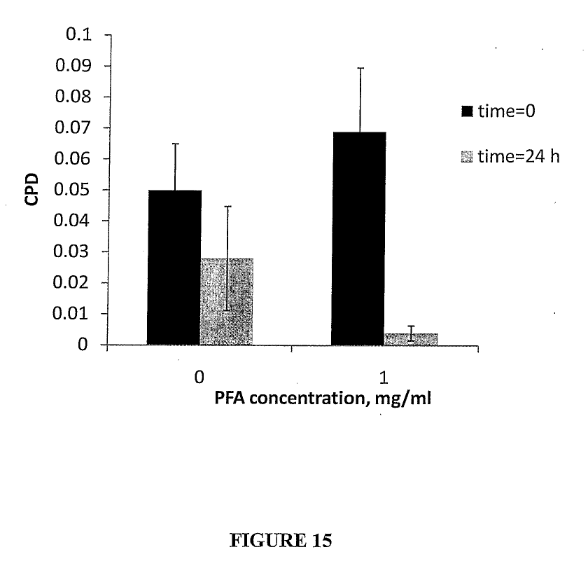

The data is shown in FIG. 15. Polyhydroxylated fatty alcohol treatment at concentration 1 μg/ml resulted in a remarkable reduction in CPD concentration in the cells 24 hours after irradiation

### Example 12

### Effect of Polyhydroxylated Fatty Alcohols on Keratinocytes and Fibroblasts Viability

HaCaT cells were grown as is written in Example—5. HaCaT cell viability was measured by MTT assay. The cells were inoculated in 96 well Microtitre plates at concentration of 1×10⁵ cells/well.

After overnight culture, 100 μl of polyhydroxylated fatty alcohol supplemented medium was added to cells and incubated for 4 h or 24 h. 100 μl of MTT (5 mg/ml) was added to each well. After 4 h of incubation at 37° C., the plate was centrifuged at 1000 rpm for 50′. The supernatant was removed and precipitate was dissolved in 100 μl of isopropanol. The O.D of the resulting solution was measured spectrophotometrically at 540 nm.

Normal human dermal fibroblasts were provided by M. Chaouat M. Sc. (Laboratory of Experimental Surgery, Hadassah Hospital, Jerusalem, Israel). Fibroblasts were obtained from healthy donors undergoing breast plastic surgery. The cells were cultured in 9-cm culture dishes, at 37° C. in 5% CO₂ in DMEM (Biological Industries, Beit Haemek, Israel) supplemented with 10% fetal calf serum, L-glutamine 2 mM and antibiotics (100 U penicillin per ml and 100 mg streptomycin per ml). The experiments were performed between passages 4 and 8.

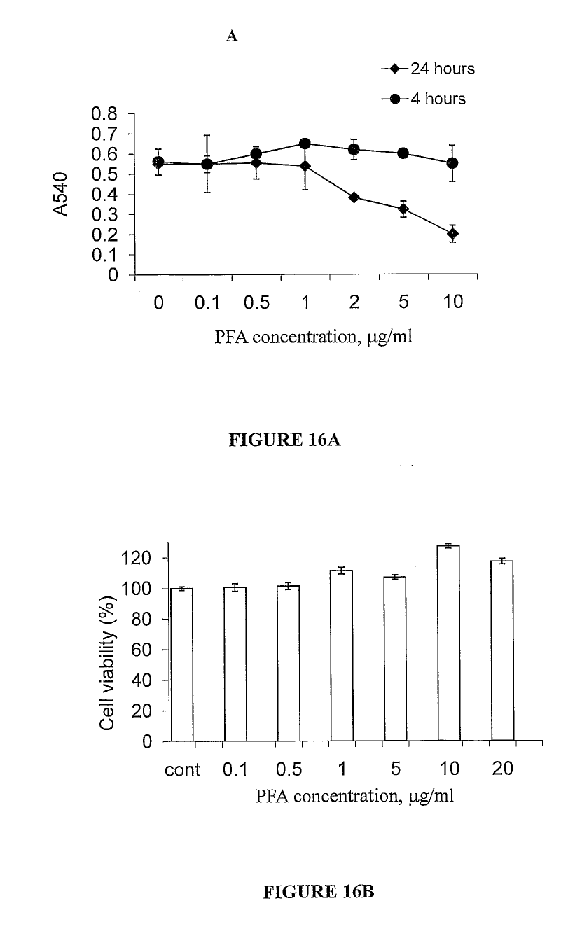

Fibroblasts viability was measured by MTT assay on 24-well plates. Short term HaCaT cell treatment (4 h) with PFA at concentration up to 10 μg/ml did not reveal an effect of PFA on cell proliferation. In contrast, long term cell treatment (for 24 h) with PFA at concentrations higher than 1 μg/ml demonstrated dose-dependent decrease of cell viability. Smaller PFA concentration still were non toxic for HaCaT cells (FIG. 16A)

No effect of PFA on fibroblast viability was detected at concentrations of up to 20 μg/ml (FIG. 16 B).

### Example 13

### Preparation of Cream Composition Based on Non-Ionic Oil in Water Emulsion Containing Polyhydroxylated Fatty Alcohols from Avocado Seeds for Cosmetic Use

The non-ionic oil in water emulsion comprised 20%-40% oil phase (w/w) of the composition. The oil phase contained wheat germ oil, polyunsaturated fatty acid, vitamin A, E and F, ascorbyl palmitate and antioxidants. The water phase was 60%-80% (w/w) and contained a mixture of water, plant extracts, humectants and non-ionic emulsifiers.

The concentration of avocado derived polyhydroxylated fatty alcohols was 0.01%-1% (w/w). Cream was prepared by inverse emulsion process by addition of the water phase to the oil phase. The polyhydroxylated fatty alcohols were pre-dissolved into the oil phase and the nonionic emulsifiers were pre-disclosed into the water phase. Both phases were preheated to 75° C. The emulsification was produced by a high speed homogenizer for 20-40 minutes, continued for another 60-120 min by a low-speed planetary mixer.

### Example 14

### Preparation of a Lipophilic Natural Oil Composition Containing Polyhydroxylated Fatty Alcohols from Avocado Seeds for Cosmetic Use

The lipophilic natural oil composition comprised a mixture of a polyhydroxylated fatty alcohols, jojoba oil, gamma linolenic acid, promigranate oil, avocado oil, vitamins A, E, and F, antioxidants and propylene glycol and dipelargonate ethoxydiaglycol.

Polyhydroxylated fatty alcohols were presented in a concentration 0.05%-0.1% (w/w). The concentration of natural vegetable oils was 70% (40%-80%) (w/w). The polypropylene glycol dipelargonate was present in a concentration of about 1.60% (2%-7%) (w/w) and ethoxydiaglycol was presented in concentration of about 0.4% (0.05%-1%) (w/w). The additional components were present in a concentration of 27.95% (10%-50%) (w/w).

### Example 15

### Clinical Toxicological Study—Repeat Insult Patch Test (RIFT)

Toxicity testing of the polyhydroxylated fatty alcohols was performed using the RIPT (Repeat Insult Patch Test) method. In RIPT, a small amount of the active compound is applied to the skin of each individual subject and monitored for its effect. Over a certain interval of time, the skin is observed, graded, and tested again.

The clinical work was performed by the HELA Skin Research Center, an independent internationally recognized testing company which specializes in the evaluation of products for the cosmetic, pharmaceutical, food supplement, raw materials, medical devices, and textile industries.

The objective of the study was to determine the irritation and/or sensitization potential of skin treated with polyhydroxylated fatty alcohols under conditions of repeated administration to the skin of human subjects. The subjects were informed of the nature of the test including possible adverse reactions. Written informed consent documents were signed by all participants prior to induction. Only subjects that were able to read, understand and follow directions were requested to participate. Prior to initiation of a test, each subject completed a medical history form. The subjects did not exhibit any physical or dermatological condition, which would preclude application of the test material(s).

The Repeat Insult (RIPT) was performed with 50 human subjects, in two phases. The first phase is the induction phase. The quantity of test material applied per test patch was approximately 0.2 mL or 0.2 g of each substance at 5% w/w. Test material(s) was placed on a 2 cm square Parke-David Readi-Bandage (occlusive) or to a 2 cm square of Webril non woven fabric affixed to Scanpor tape (semi-occlusive) or equivalent coverings. The patches were applied to the subject's back between the scapulae and waist. The subjects removed the patches 24 hours after each application. 24 hour rest periods followed each removal. Prior to each reapplication, site(s) were evaluated by an experienced and certified HELA staff member.

This procedure was repeated until 9 applications of the test material(s) were made. Skin responses were evaluated according to the following scoring:

0) No visible reaction.
?) Doubtful reaction: faint, minimal erythema, no infiltration'
1) Weak positive reaction: erythema, infiltration' discrete papules'
2) Strong positive reaction: erythema, infiltration, papules, discrete vesicles.
3) Extra positive reaction: intense erythema, infiltration, coalescing vesicles, bulbous reaction.
IR) Irritant reaction: discrete erythema without infiltration/patchy follicular erythema/hemorrhagic and follicular pustules.

### NT) Not Tested.

If a subject developed a positive reaction of a 2-level or greater during the induction phase, the patch was applied to a fresh adjacent site for the next application. If a 2 or greater reaction occurred at the second site or application, no further applications of the reactive test material were made for the remainder of the induction phase.

The second phase is the challenge phase. Ten to 21 days after application of the final induction patch, challenge patch(es) are applied to previously unpatched (virgin) sites, adjacent to the original induction patch sites. The challenge sites are scored 24 to 48 hours after application. The subjects were asked to report of any delayed reactions, which might occur after the final challenge patch reading.

### Results

The test was performed on 50 volunteers: 7 males and 43 females. The ages of the subjects were relatively evenly distributed between 15 to 65 years of age.

No skin responses were found in any of the 50 volunteers during or after any of the applications of the test material. The material may thus be considered to be non-irritating and non-toxic to the skin.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Citation or identification of any reference in this application shall not be construed as an admission that such reference is available as prior art to the present invention.

To the extent that section headings are used, they should not be construed as necessarily limiting.

1. A topical cosmetic composition comprising polyhydroxylated fatty alcohols or derivatives thereof in an amount effective to improve the aesthetic appearance of human skin; and a cosmetically acceptable carrier; wherein said polyhydroxylated fatty alcohols or derivatives thereof comprise a backbone of from C13 to C25 carbons, with hydroxyl groups present at C1, C2 and C4, optionally with one of said hydroxyl groups present at C1, C2 and C4 in oxidized form, and at least one unsaturated carbon bond; wherein said at least one unsaturated carbon bond is present between the last two carbons of the backbone; wherein said composition is substantially free of furan lipids.

2. A method for improving the aesthetic appearance of human skin following exposure to ultraviolet radiation, the method comprising applying to the skin a composition comprising polyhydroxylated fatty alcohols or derivatives thereof in a cosmetically effective amount, in a cosmetically acceptable carrier; wherein said polyhydroxylated fatty alcohols or derivatives thereof comprise a backbone of from C13 to C25 carbons, hydroxyl groups present at one or more of C1, C2 or C4, and at least one unsaturated carbon bond; wherein said at least one unsaturated carbon bond is present between the last two carbons of the backbone; wherein said composition is substantially free of furan lipids.

3. (canceled)

4. (canceled)

5. (canceled)

6. (canceled)

7. The composition of claim 1, wherein said polyhydroxylated fatty alcohols are isolated natural polyhydroxylated fatty alcohols.

8. The composition of claim 7, wherein said isolated natural polyhydroxylated fatty alcohols are isolated from a fruit or vegetable source.

9. The composition of claim 8, wherein said fruit or vegetable source is selected from the group consisting of avocado fruit and avocado seed.

10. The composition of claim 1, wherein said derivative of natural polyhydroxylated fatty alcohols substantially comprises an acetylated derivative.

11. The composition of claim 10, wherein said polyhydroxylated fatty alcohols or derivatives thereof are isolated in substantially pure form.

12. The composition of claim 1, wherein said polyhydroxylated fatty alcohols or derivatives thereof are synthetically prepared.

13. The composition of claim 1, wherein said polyhydroxylated fatty alcohols or derivatives thereof are present at a purity of from about 80% to about 95% w/w of the isolated material.

14. The composition of claim 1, wherein said polyhydroxylated fatty alcohols are present in a concentration of from about 0.001% to about 20% w/w of the total composition.

15. (canceled)

16. (canceled)

17. The composition of claim 1, wherein said polyhydroxylated fatty alcohols or derivatives thereof or combination thereof are selected from the group consisting of, 1-Acetoxy-2,4-dihydroxy-16-heptadecene or 1,2-dihydroxy-4-acetoxy-16-heptadecene, 1-Acetoxy-2,4-dihydroxy-16-heptadecyne or 1,2-dihydroxy-4-acetoxy-16-heptadecyne

18. (canceled)

19. (canceled)

20. The composition of claim 1, wherein said composition comprises an additional compound selected from the group consisting of anesthetics; anti-allergenic; antimicrobials; antifungals; antiseptics; chelating agents; colorants; depigmenting agents; emollients; exfollients; fragrances; emulsifiers; humectants; insect repellents; lubricants; moisturizers; preservatives; skin penetration enhancers; stabilizers; sunscreens; surfactants; thickeners; viscosity modifiers; and vitamins.

21. The composition of claim 1, wherein said composition is in a form selected from the group consisting of aerosol, cake, cream, ointment, emulsion, essence, foam, gel, lotion, mousse, paste, patch, pencil, serum, solution, towelette, mask, body wrap, spray and stick.

22. The composition of claim 21, wherein said emulsion is an oil in water emulsion and wherein said oil in water emulsion comprises from about 30% to about 80% w/w oil phase.

23. (canceled)

24. The composition of claim 22, wherein said oil phase is selected from the group consisting of natural vegetable oils, polyunsaturated fatty acids, vitamin A, E, and F, ascorbyl palmitate, anti-oxidants and other suitable components, or mixtures thereof.

25. (canceled)

26. (canceled)

27. (canceled)

28. The method of claim 2, further comprising reducing external signs of UV damage or preventing UV damage to human skin by applying said composition.

29. (canceled)

30. The method of claim 2, further comprising reducing external signs of UV-induced aging in human skin or for preventing external signs of UV-induced aging in human skin by applying said composition.

31. (canceled)

32. The method of claim 30, wherein said external sign of aging comprises the appearance of pigmented spots.

33. The method of claim 30, wherein said pigmented spots are selected from the group consisting of freckles, melasma, and solar lentigines.

34. The method of claim 33, wherein said external signs of UV-induced aging are selected from the group consisting of fragile skin, sagging skin, fine lines, wrinkles, thinning skin, lack-luster skin, fatigued skin, and dry skin.

35-43. (canceled)
